(19) United States
(12) Patent Application Publication
(10) Pub. No.: US 2019/0374631 A1
(43) Pub. Date: Dec. 12, 2019

(54) METHODS OF PRIMING A SUS' IMMUNE SYSTEM

(71) Applicant: APTIMMUNE BIOLOGICS, INC., St. Louis, MO (US)

(72) Inventors: Aaron Gilbertie, Greentown, IN (US); Steven Berger, Two Harbors, MN (US); Federico Zuckermann, Champaign, IL (US)

(21) Appl. No.: 16/312,946

(22) PCT Filed: Jun. 23, 2017
(86) PCT No.: PCT/US2017/039110

§ 371 Date: Dec. 21, 2018

Related U.S. Application Data
(60) Provisional application No. 62/354,534, filed on Jun. 24, 2016.

(51) Int. Cl. A61K 39/04; A61K 39/39; A01K 67/02; A61P 37/04

(52) U.S. Cl. CPC A61K 39/04; A61K 39/39; A61K 2039/52; A61P 37/04; A01K 67/02

(57) ABSTRACT

Methods of priming a Sus' immune system are disclosed. The methods comprise administering an effective amount of a Mycobacterial whole cell lysate to a Sus within an effective period of time after the Sus is born.

### BACKGROUND

Respiratory infections are a major cause of mortality among piglets of nursery age, which ranges from about 19 to about 68 days resulting in significant economic loses to the pork industry.¹ For example, porcine reproductive and respiratory syndrome (PRRS) is a chronic viral disease of pigs worldwide. PRRS is endemic in most pork-producing countries, and it is responsible for major economic losses to the swine industry, with an estimated annual loss of $664 million in the US.² Clinical signs of PRRS comprise respiratory and reproductive dysfunction, and the causal agent is the PRRS virus (“PRRSV”).³ PRRSV establishes disease by modulating the pig immune system from as early as two days and continues for several weeks post-infection.⁴

Vaccination of piglets is a strategy commonly used to combat respiratory infections; however, attempts to attain neonatal protection with a vaccination approach are considered ineffective.⁵ The challenge for the successful immunization in neonates arises as a consequence of the immaturity of the neonatal immune system, which is known to have a limited capacity to make cell-mediated immune responses that involve cytotoxic T cells as well as IFN-gamma producing T cells (i.e., T helper (Th)1 cells). As a result, the defense against intracellular pathogens including viruses is ineffective.⁶ Representative reports of the capabilities of neonatal antigen presenting cells (“APCs”), lymphocytes, and other cells of the innate immune system implicated in the development of adaptive immunity indicate a limited response to mitogens, differences in cytokine profiles, lack of development of anatomic structures, and differences in expression of membrane receptors that are necessary for the development of proper and protective adaptive immune response.⁷

The inadequacy of the innate immune system in the newborn, which is necessary to enable a proper adaptive immune response to vaccination, is manifested by an impaired vaccine-induced antibody response in terms of both quantity and quality.⁸ This condition is demonstrated by differences in the magnitude of antibody response to vaccination against swine influenza virus (“SIV”) depending on the age at which newborn swine are vaccinated. Piglets that were vaccinated for the first time at 1 week of age developed lower maximum antibody titers after the second vaccination, and become seronegative earlier than pigs that were vaccinated for the first time at 4 or 8 weeks of age.⁹ Similarly, piglets vaccinated against Porcine Circovirus Disease (“PCVD”) at 3 weeks of age were better protected against this virus than pigs vaccinated at 1 week of age.^{m} Thus, one of the major challenges in neonatal swine vaccinology is that biologics are unable to elicit adequate protective immunity in the early life period because the naïve (unprimed) state of the innate immune system fails to provide adequate signaling for T cell activation as well as the optimal cytokine milieu to enable the development of an adaptive immune response of sufficient quality and strength to provide anti-microbial protective immunity.

Because the immune system of a newborn swine is not sufficiently mature, it requires several weeks after birth to be ready to develop an adequate adaptive immune response to the antigenic stimuli provided by a vaccine. As a result, the newborn lung is heavily dependent on the innate immune system for protection against airborne pathogens. Currently there are no fully effective vaccines or therapies for viral or bacterial respiratory infections of swine. However, different approaches have been attempted to address these problems.

One approach to try to address these problems is to administer innocuous but immune-stimulating materials to activate the neonate's innate immune pathways, which, by promoting its development, would accelerate its maturation and functionality. The strategies that have been explored to promote the development of the innate immune system of newborn swine include dietary supplementation with beta-glucan, a component of yeast cell wall, or with different plant extracts.¹¹ Although results indicating the stimulation of a systemic immune-stimulating effect have been reported, dietary supplementation does not, however, directly target for its effect in cells of the innate immune system that reside in the respiratory tract.

In addition to dietary supplementation, another approach is to directly prime cells of the innate immune residing in the respiratory tract. The cells of the innate immune system, including, for example, macrophages and dendritic cells, play a direct role in mediating protective immunity or as antigen presenting cells (“APC”). In humans, Bacillus Calmette-Guerin (“BCG”), a live bacterium, is regularly given at birth in humans, which is capable of inducing strong Thl-type immune responses. Without being bound by any theory, the effectiveness of the BCG vaccine is believed to be due to the ability of this microbe to engage multiple toll like receptors (“TLRs”) expressed by APCs which as a result produce pro-inflammatory cytokines and promote the development of Thl immunity.

Another approach to try to address these problems is the administration by injection of microbial products, which are used as immune-modulators, including, but not limited to, heat killed or formaldehyde treated suspensions of Propionibacterium acnes, microbial polysaccharides, lipopolysaccharides, protein-bound polysaccharides, muramyl-dipeptide, lipid A, and deproteinized and delipidated Mycobacterium phlei cell wall extract (MCWE). For example, U.S. Pat. No. 4,744,984 (Vetrepharm Research, Inc.) discloses methods of treating a viral infection in animals and humans comprising the step of injecting an animal or human with a deproteinized bacterial cell wall suspension in an oil and water emulsion, and the bacterial cell wall suspension can be derived from a Mycobacterium species. U.S. Pat. No. 5,759,554 (Vetrepharm Research, Inc.) discloses methods of stimulating the immune system in a human or animal comprising administering to the human or animal an aqueous suspension of an insoluble bacterial cell wall fraction that does not contain oil, and the insoluble cell wall fraction is prepared from Mycobacterium species and treated to extract lipids from the fraction. U.S. Pat. No. 6,890,541 (Bioniche Life Sciences, Inc.) discloses methods for activating the immune system of a newborn animal to enhance production performance of the animal comprising administering to the newborn animal Mycobacterium cell wall extract.

Unfortunately, however, the methods disclosed in U.S. Pat. Nos. 4,744,984, 5,759,554, and 6,890,541 have significant shortcomings. One shortcoming is that administration of cell wall suspensions, cell wall fractions or cell wall extracts are potentially not as effective as other strategies. For example, the administration of cell wall suspensions, cell wall fractions or cell wall extracts is limited to only cell wall core components and is unlikely to include any of the structural components that are present in the outer leaflet of the Mycobacterial envelope. The cell wall structure is only a small fraction of the Mycobacterial envelope. Without being bound by any theory, it is hypothesized that administering the core cell wall core components in combination with the structural components present in the outer leaflet of the Mycobacterial envelope has an additive and possibly synergistic effect on the stimulation of a newborn animal's immune system compared to administering only cell wall core components.

Another shortcoming is that in some embodiments in U.S. Pat. Nos. 4,744,984, 5,759,554, and 6,890,541 during the process of cell wall extraction and fractionation the cell walls are delipidated, in which case at least two major components of the Mycobacterial envelope, namely TDM and LAM which are present in the outer leaflet of the envelope and are known to have immunostimulating activity, are most likely removed during delipidation.¹² The components remaining after delipidation consist of the cell wall core structure, which, although an important fraction of the Mycobacterial envelope, is missing prominent Mycobacterial components of the outer leaflet such as, for example, TDM and LAM, that are known to have the ability to activate macrophages and thus trigger innate host responses, e.g., the production of inflammatory cytokines.

A further shortcoming is that administering only cell wall core components is inconvenient. For example, isolating or extracting cell wall core components can be time consuming, requiring several steps. Yet another potential shortcoming is that cell wall core components are insoluble in aqueous formulations and require lipids or oil based emulsions for delivery.

Although strategies are available for addressing the above-mentioned problems regarding the problem of respiratory infections causing major mortality among piglets, such strategies may be inconvenient, have drawbacks, and be less effective than other strategies. Accordingly, there exists a need for alternatives for combating respiratory infections in piglets. Preferably, such alternatives are more effective than other strategies and decrease the inconvenience and drawbacks of one or more of the current approaches.

### SUMMARY

The present disclosure addresses the problems described above by providing effective and efficient methods of priming a Sus' immune system that exhibit desirable properties and provide related advantages as well. In some embodiments of the present disclosure, the methods comprise administering an effective amount of a Mycobacterial whole cell lysate to the Sus within an effective period of time after the Sus is born.

Another aspect of the present disclosure provides a Mycobacterial whole cell lysate for use in priming a Sus' immune system. In some embodiments of the present disclosure, the Mycobacterial whole cell lysate for use in priming a Sus' immune system comprises administering an effective amount of the Mycobacterial whole cell lysate to the Sus within an effective period of time after the Sus is born.

Another aspect of the present disclosure provides a use of a Mycobacterial whole cell lysate for the manufacture of a medicament for use in priming a Sus' immune system. In some embodiments of the present disclosure, the use comprises administering an effective amount of the Mycobacterial whole cell lysate to the Sus within an effective period of time after the Sus is born.

The present disclosure provides several advantages compared to other approaches in the art that have been utilized. One advantage of a method according to an embodiment is that administration of a Mycobacterial whole cell lysate contains most, if not all, of the structural components of the Mycobacterial envelope. Therefore, administering a Mycobacterial whole cell lysate, which includes the structural components of the Mycobacterial envelope rather than only cell wall core components, has an additive and possibly synergistic effect on the stimulation of a Sus' immune system compared to administering only cell wall components.

An advantage of a method according to another embodiment is that the Mycobacterial whole cell lysate utilized in accordance with the present disclosure would not be delipidated. Thus, unlike the lipid extraction procedures that are employed when preparing cell wall suspensions, cell wall fractions or cell wall extracts, the cell wall core components that have potent immunostimulating activity in the Mycobacterial whole cell lysate of the present disclosure would not be lost.

An advantage of a method according to another embodiment is that a Mycobacterial whole cell lysate is easier to prepare than cell wall suspensions, cell wall fractions or cell wall extracts, which reduces the inconvenience and inefficiencies of other approaches. For example, preparing a Mycobacterial whole cell lysate, containing all or substantially all of the structural components of the Mycobacterial envelope, requires fewer steps and less time than isolating or extracting cell wall components.

An advantage of a method according to another embodiment is that the process steps required to prepare a Mycobacterial whole cell lysate are readily scalable compared to traditional industrial fermentation facilities and equipment unlike the process steps required to prepare cell wall suspensions, cell wall fractions or cell wall extracts.

### BRIEF DESCRIPTION OF DRAWINGS

The above-mentioned aspects of embodiments will become more apparent and will be better understood by reference to the following description of the embodiments taken in conjunction with the accompanying drawings, wherein:

FIG. 1 shows the TNF-alpha response of alveolar macrophages to stimulation with lipopolysaccharide (“LPS”) compared to a crude whole cell lysate of Mycobacterium smegmatis.

FIG. 2 shows the kinetics of the TNF-alpha response of porcine alveolar macrophages to stimulation with a crude whole cell lysate of Mycobacterium smegmatis and influence of the culture medium used to grow the bacteria on the potency of the WCL. Three different types of culture medium, 7H9, GAS or NB, were used to culture Mycobacteria to prepare the bacterial cell mass used to obtain the crude whole cell lysate.

FIG. 3 shows that the potency (as indicated by the 50%-effective dose) of the Mycobacterium smegmatis WCL can be affected by the type of growth media that is used to culture the Mycobacterium smegmatis in order to prepare the bacterial cell mass to prepare the WCL.

FIG. 4 shows the relative potency of crude Mycobacterium smegmatis WCL as compared to (1) a commercial preparation of lipoarabinomannan (“LAM-MS”) from Mycobacterium smegmatis, and (2) a commercial preparation of Mycobacterium phlei cell wall extract.

FIG. 5 shows a TNF-alpha Stimulation enhanced effect in pigs from administration of Mycobacterium smegmatis WCL.

FIG. 6 shows a Natural Killer Subpopulation enhanced effect in pigs from administration of Mycobacterium smegmatis WCL.

FIG. 7 shows a B-Cell Subpopulation enhanced effect in pigs from administration of Mycobacterium smegmatis WCL.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

Since the respiratory tract is a major target for disease susceptibility in newborn swine and because the structural components of Mycobacteria are known to engage several pathways of the innate immune system, the present disclosure addresses this problem by the delivery of a Mycobacterium whole cell lysate to directly prime the innate immune system of the respiratory tract of a newborn swine and enhance its defense mechanisms at this critical port of entry for respiratory pathogens.

The immunostimulating activity of the various structural components of the Mycobacterial envelope have been recognized for some time.¹³ The Mycobacterial cell envelope is complex and consists of a thick waxy mixture of lipids, polysaccharides, glycolipids, and mycolic acids, which are arranged in layers.¹⁴ These layers first consist of an inner membrane (“IM”) comprised of conventional polar lipids that form a typical membrane bilayer, and include, as a significant component, phosphatidylinositol mannosides (“PIMs”). Covering the IM is the peptidoglycan-arabinogalactan (“AGP”) complex that forms a scaffold consisting of a helical peptidoglycan (“PG”) moieties network interspersed with helical galactan (polymers of galactose) that provide anchorage to the polysaccharide arabinan. When the galactan and arabinan polysaccharides are combined, the combined structure constitutes the arabinogalactan (“AG”) component of the envelope.¹⁵ In turn, the distal arabinose moieties of the AG unit provide anchorage via covalent links to mycolic acids. This lower segment of the cell wall is termed the cell wall core, namely the mycolyl arabinogalactan-peptidoglycan (“mAGP”) complex.¹⁶ The Mycobacterial envelope is finally covered with an upper layer that is composed of extractable lipids, which is known in the art as the upper segment, the outer leaflet or the outer membrane. The extractable lipids in this outer leaflet of the envelope are composed of different types of lipids, including fatty acids, lipooligosaccharides (“LOS”), triacyl lipopeptides, glycopeptidolipids (“GPL”), trehalose dimycolate (“TDM”) and lipoglycans, namely lipoarabinomannan (“LAM”).¹⁷

The components of the OM exist in the Mycobacterial cell wall as “free” lipids (i.e., as solvent-extractable lipids that are not covalently linked to the underlying peptidoglycan-arabinogalactan (“AGP”) complex.¹⁸ The immune stimulating activity of the TDM and LAM have been extensively studied. TDB binds the C-Type lectin, Mincle (macrophage-inducible C-type lectin).¹⁹ Upon TDB recognition, C-Type lectin, Mincle interacts with the Fc receptor common γ-chain (“FcRγ”), which triggers intracellular signaling through Syk leading to CARDS-dependent NF-κB activation. LAMs are lipoglycans restricted to the Mycobacterium genus that act as potent modulators of the host immune response and are found in the envelope of all Mycobacteria species, such as the pathogenic strains M. tuberculosis and M. leprae, the vaccine strain, M. bovis BCG, the opportunistic strains M. avium and M. foruitum, and the nonpathogenic strain M. smegmatis. LAM display different immunomodulatory effects depending on their structure. PILAM, which are phosphoinositol-capped LAM and found in nonpathogenic species (M. smegmatis), are proinflammatory molecules whereas ManLAM, which are mannose-capped LAM and found in pathogenic species (M. tuberculosis), are anti-inflammatory molecules.²⁰ PILAM activates macrophages in a TLR2-dependent manner that seems to involve other TLRs but not TLR4.²¹

To define the Mycobacterial structural components that have immunostimulating activity, various techniques have been employed to fractionate and purify these components such that these components can be individually studied. Most of these techniques are based on mechanical disintegration of the bacteria followed by differential centrifugation. After fracturing the bacteria by mechanical means, the resulting components can be separated by differential centrifugation. Centrifugation of the WCL at a low speed (3,000×g, where g is gravitational field of strength) results in the elimination of unbroken cells with all other structural components of the bacteria remaining in the suspension. On the other hand, centrifugation of the WCL at a high speed 27,000 g results in the separation of the cell wall, which would pellet down after centrifugation while the membrane and cytosol components remain suspended in the supernatant.²² The resultant cell wall pellet contains the mAGP complex as well as the associated LAM.²³ This type of composition would occur only in WCL preparations that have not been deliberately delipidated at any point during the bacterial fractionation procedure. Otherwise, after delipidation, the extractable lipid molecules that normally compose the outer leaflet, such as TDM and LAM, are lost during the extraction procedure. Indeed, delipidated Mycobacterium smegmatis have been shown to be unable to be recognized by the macrophage receptor Mincle (macrophage inducible C-type lectin), which recognizes mycobacterial TDM and is one of the free lipids present on the outer leaflet of the Mycobacterial envelope.²⁴ Thus, a crude whole cell lysate of Mycobacteria would be expected to have most if not all of the macromolecules known to be present in the Mycobacterial envelope of this type of bacteria.

The structural components of Mycobacteria are recognized by a number of host receptors expressed in myeloid cells, including most prominently macrophages and dendritic cells, Toll-like receptors, nucleotide-binding oligomerization domain (NOD)-like receptors (“NLRs”), C-type lectin receptors like Minicles and the mannose receptor (“CD207”), the dendritic cell-specific intercellular adhesion molecule-3 grabbing nonintegrin (“DC-SIGN.CD209”), and Dectin-1.²⁵ Most TLR-dependent signals initiated by Mycobacteria are positive, leading to activation of the inflammatory and antimicrobial innate immune responses. For example, the Phosphoinositol-capped LAM from fast-growing and avirulent species, such as Mycobacterium smegmatis, are pro-inflammatory molecules stimulating the production by macrophages of tumor necrosis factor (TNF)-alpha and IL-12.²⁶ Whereas most bacteria produce N-acetyl MDP, Mycobacteria produce an unusual modified form of MDP called N-glycolyl MDP, which is a very potent inducer of type I interferon (“IFN”) and has been shown to be very effective at providing protection against influenza virus infection.²⁷ In addition, as described above, the Mycobacterial envelope outer leaflet contains a wide array of chemically diverse lipids and glycolipids that likely mediate specific host interactions and have been shown to possess potent biologically activity against eukaryotic cells in vitro.²⁸

“Priming a Sus' immune system” refers to stimulating and/or activating the immune system of a Sus and includes causing an immune response by cells of the Sus' immune system. An “immune response” is a response of a cell of the immune system, such as, for example, a B cell, T cell, monocyte or the like, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”). An immune response can also include the innate response. In some embodiments, priming a Sus' immune system comprises priming macrophages. In some embodiments, priming a Sus' immune system comprises priming alveolar macrophages. In some embodiments, the primed alveolar macrophages exhibit enhanced production of TNF-alpha in response to a stimulus. If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo. In some embodiments, the immune response is localized. In some embodiments, the immune response is systemic.

In some embodiments of the present disclosure, “priming a Sus' immune system” includes “priming a Sus' immune system for vaccination.” It is envisioned that the vaccination can be against any type of virus, bacteria, fungi, protozoa, or other parasites that can infect a Sus. A non-limiting list of the viruses that the vaccinations can target include without limitation PRRSV, swine influenza virus, porcine circovirus, porcine parvovirus (“PPV”), transmissible gastroenteritis (“TGE”) virus, porcine epidemic diarrhea virus (“PEDV”), porcine rotavirus, swine paramyxovirus, pseudorabies virus, African swine fever virus (“ASFV”), Classical swine fever virus (“CSF”), swine coronavirus family, porcine torque teno virus, porcine bocavirus, porcine torovirus, swine hepatitis E virus, porcine endogenous retrovirus, porcine lymphotropic herpesvirus, porcine sapovirus, porcine pestivirus, Nipah virus, Bungowannah virus, Menangle virus, and delta coronavirus.

A non-limiting list of the bacteria that the vaccinations can target include without limitation Mycoplasma suis; Pasteurella haemolytica; Haemophilus somnus; Brucella abortus; chlamydia; anaplasma; mycoplasma; Actinobacillus pleuropneumoniae; Actinobacillus suis and equuli; Bordetella bronchiseptica; Brucella suis; Campylobacter coli, jejunum, hyointestinalis; Escherichia coli (E. coli); Haemophilus parasuis; Klebsiella species; Lawsonia intracellularis; Leptospira pomona; Leptospira bratislava/muenchen; Leptospira icterohaemorrhagiae; Pastueurella multocida (toxigenic); Pasteurella multocida (non-toxigenic); Salmonella choleraesuis; Salmonella typhimurium, derby, and others; Brachyspira pilosicoli; Brachyspira hyodysenteriae; Brachyspira (weak haemolytic sp); Yersinia species; Actinomyces (Corynebacterium) pyogenes; Bacillus anthracis; Brucella suis; Chlamydia psittaci; Clostridium novyi; Clostridium perfringens; Clostridium tetani; Actinobaculum (Corynebacterium, Eubacterium) suis; Eperythrozoon suis; Enysipelothrix rhusiopathia; Listeria monocytogenes; Mycobacterium avium/intracellulare; Mycoplasma hyopneumoniae; Mycoplasma flocculare; Mycoplasma hyorhinis; Mycoplasma hyosynoviae; Staphyloccus hyicus; other Staphylococci; Streptococcus suis type 1; Streptococcus suis type 2, type 15; and other types of Streptococcus.

As used herein, “Sus” refers to any animal, wild or domestic, that is a member of the biological family Suidae, including without limitation Babyrousa babyrussa or Golden Babirusa, Babyrousa celebensis or Sulawesi Babirusa, Babyrousa togeanensis or Togian Babirusa, Hylochoerus meinertzhageni or Giant Forest Hog, Phacochoerus aethiopicus or Cape, Somali or Desert Warthog, Phacochoerus africanus or Common Warthog, Porcula salvania or Pygmy Hog, Potamochoerus larvatus or Bushpig, Potamochoerus porcus or Red River Hog, Sus ahoenobarbus or Palawan Bearded Pig, Sus barbatus or Bearded Pig, Sus bucculentus or Vietnamese Warty Pig, Sus cebifrons or Visayan Warty Pig, Sus celebensis or Celebes Warty Pig, Sus heureni or Flores Warty Pig, Sus oliveri or Mindoro Warty Pig, Sus philippensis or Philippine Warty Pig, Sus scrofa or Wild Boar or Domestic Pig, Sus verrucosus or Javan Warty Pig, and any other boar, sow, piglet, farrow, shoat, gilt, barrow, hog, swine or porcine of either sex or any age.

The methods of the present disclosure utilize a Mycobacterial whole cell lysate. As used herein, “whole cell lysate,” which is commonly abbreviated as “WCL,” has the same meaning as commonly understood by one of ordinary skill in the art to which the present disclosure belongs. In some embodiments, the Mycobacterial whole cell lysate is a crude Mycobacterial whole cell lysate. In some embodiments, crude Mycobacterial whole cell lysate includes, for example, lysed Mycobacterium cells from which no structural components have been removed or subjected to fractionation, other than to remove unfractured cells, and no other partition, extraction or separation of either a physical or chemical nature. In some embodiments, the mycobacterial whole cell lysate includes lysed cells that are dead and can no longer replicate but contain all of the components of the pre-lysed cells. In some embodiments, the whole cell lysate is a non-denatured supernatant of WCL. In some embodiments, the Mycobacterial whole cell lysate is an adjuvant.

In some embodiments of the present disclosure, the Mycobacterial whole cell lysate has not undergone purification. In some embodiments of the present disclosure, the Mycobacterial whole cell lysate has undergone purification. As used herein, “purification” refers to the process of removing components that are not desired from a Mycobacterial whole cell lysate. Purification does not require that all traces of the undesirable component be removed from the Mycobacterial whole cell lysate. Purification techniques include without limitation cell fractionation, centrifugation, dialysis, ion-exchange chromatography, size-exclusion chromatography, and affinity-purification or precipitation. In some embodiments of the present disclosure, the Mycobacterial whole cell lysate is unfractionated. In some embodiments of the present disclosure, the Mycobacterial whole cell lysate is not delipidated. In some embodiments of the present disclosure, the Mycobacterial whole cell lysate is not deproteinized. In some embodiments, the Mycobacterial whole cell lysate is administered alone. In some embodiments of the present disclosure, the Mycobacterial whole cell lysate is administered with one or more suitable vaccines against swine viral disease.

The Mycobacterial whole cell lysate utilized in the methods of the present disclosure may be prepared from any Mycobacterium. As used herein, “Mycobacterium” refers to any prokaryote that is from the family Mycobacteriaceae or genus Mycobacterium. A non-limiting list of Mycobacteria that can be utilized in the methods of the present disclosure include without limitation Mycobacterium bovis, Mycobacterium africanum, Mycobacterium microtti, Mycobacterium tuberculosis, Mycobacterium canettii, Mycobacterium marinum, Mycobacterium avium intracellulare, Mycobacterium leprae, Mycobacterium lepraemurium, Mycobacterium paratuberculosis, Mycobacterium ulcerans, Mycobacterium smegmatis, Mycobacterium xenopi, Mycobacterium chelonei, Mycobacterium fortuitum, Mycobacterium farcinogenes, Mycobacterium flavum, Mycobacterium haemophitum, Mycobacterium kansasii, Mycobacterium phlei, Mycobacterium scrofulaceum, Mycobacterium senegalense, Mycobacterium simiae, Mycobacterium thermoresistible, Mycobacterium vaccae, Mycobacterium porcinum, Mycobacterium abscessu, Mycobacterium peregrinum, Mycobacterium phlei, Mycobacterium alvei, and Mycobacterium xenopi.

The Mycobacterial whole cell lysate utilized in the methods of the present disclosure may be administered using any applicable route that would be considered by one of ordinary skill, including without limitation oral, intravenous (“IV”), subcutaneous (“SC”), intramuscular (“IM”), intraperitoneal, intradermal, intraocular, intrapulmonary, intranasal, transdermal, subdermal, topical, mucosal, nasal, impression into skin, intravaginal, intrauterine, intracervical, and rectal. In some embodiments of the present disclosure, the intranasal route of administration comprises intranasal drops. In some embodiments of the present disclosure, the intranasal route of administration comprises intranasal aerosol delivery. In some embodiments of the present disclosure, intranasal aerosol delivery comprises nasal spray delivery.

In carrying out the methods of the present disclosure, an effective amount of Mycobacterial whole cell lysate is administered to a Sus. The term “effective amount,” in the context of administration, refers to the amount of Mycobacterial whole cell lysate that when administered to a Sus is sufficient to prime a Sus' immune system. Such an amount should result in no or few adverse events in the treated Sus. Similarly, such an amount should result in no or few toxic effects. As those familiar with the art will understand, the amount of Mycobacterial whole cell lysate will vary depending upon a number of factors, including without limitation the type of Sus being treated, the Sus' age, size, weight, and general physical condition, and the dosing regimen.

In some embodiments of the present disclosure, an effective amount of the Mycobacterial whole cell lysate to be delivered to the Sus can be quantified by determining micrograms of Mycobacterial whole cell lysate per kilogram of Sus body weight. In some embodiments of the present disclosure, the amount of Mycobacterial whole cell lysate administered to the Sus is from about 0.00001 to about 1000 μg of Mycobacterial whole cell lysate per kg of Sus body weight. In some embodiments of the present disclosure, the amount of Mycobacterial whole cell lysate administered to the Sus is from about 1 to about 600 μg of Mycobacterial whole cell lysate per kg of Sus body weight. In some embodiments of the present disclosure, the amount of Mycobacterial whole cell lysate administered to the Sus is from about 1 to about 500 μg of Mycobacterial whole cell lysate per kg of Sus body weight. In some embodiments of the present disclosure, the amount of Mycobacterial whole cell lysate administered to the Sus is from about 100 to about 500 μg of Mycobacterial whole cell lysate per kg of Sus body weight. In some embodiments of the present disclosure, the amount of Mycobacterial whole cell lysate administered to the Sus is from about 100 to about 300 μg of Mycobacterial whole cell lysate per kg of Sus body weight. In some embodiments of the present disclosure, the amount of Mycobacterial whole cell lysate administered to the Sus is from about 1 to about 100 μg of Mycobacterial whole cell lysate per kg of Sus body weight. In some embodiments of the present disclosure, the amount of Mycobacterial whole cell lysate administered to the Sus is from about 1 to about 75 μg of Mycobacterial whole cell lysate per kg of Sus body weight. In some embodiments of the present disclosure, the amount of Mycobacterial whole cell lysate administered to the Sus is from about 1 to about 50 μg of Mycobacterial whole cell lysate per kg of Sus body weight. In some embodiments of the present disclosure, the amount of Mycobacterial whole cell lysate administered to the Sus is from about 1 to about 25 μg of Mycobacterial whole cell lysate per kg of Sus body weight. In some embodiments of the present disclosure, the amount of Mycobacterial whole cell lysate administered to the Sus is from about 25 to about 50 μg of Mycobacterial whole cell lysate per kg of Sus body weight.

In some embodiments of the present disclosure, an effective amount of the Mycobacterial whole cell lysate to be delivered to the Sus can be quantified by determining micrograms of Mycobacterial whole cell lysate per milliliter of a pharmaceutically acceptable carrier. In some embodiments of the present disclosure, the amount of Mycobacterial whole cell lysate administered to the Sus is from about 0.0001 to about 1000 μg of Mycobacterial whole cell lysate per mL of a pharmaceutically acceptable carrier per dose. In some embodiments of the present disclosure, the amount of Mycobacterial whole cell lysate administered to the Sus is from about 1 to about 1000 μg of Mycobacterial whole cell lysate per mL of a pharmaceutically acceptable carrier per dose. In some embodiments of the present disclosure, the amount of Mycobacterial whole cell lysate administered to the Sus is from about 1 to about 500 μg of Mycobacterial whole cell lysate per mL of a pharmaceutically acceptable carrier per dose. In some embodiments of the present disclosure, the amount of Mycobacterial whole cell lysate administered to the Sus is from about 25 to about 500 μg of Mycobacterial whole cell lysate per mL of a pharmaceutically acceptable carrier per dose. In some embodiments of the present disclosure, the amount of Mycobacterial whole cell lysate administered to the Sus is from about 50 to about 500 μg of Mycobacterial whole cell lysate per mL of a pharmaceutically acceptable carrier per dose. In some embodiments of the present disclosure, the amount of Mycobacterial whole cell lysate administered to the Sus is from about 50 to about 400 μg of Mycobacterial whole cell lysate per mL of a pharmaceutically acceptable carrier per dose. In some embodiments of the present disclosure, the amount of Mycobacterial whole cell lysate administered to the Sus is from about 50 to about 250 μg of Mycobacterial whole cell lysate per mL of a pharmaceutically acceptable carrier per dose. In some embodiments of the present disclosure, the amount of Mycobacterial whole cell lysate administered to the Sus is from about 50 to about 300 μg of Mycobacterial whole cell lysate per mL of a pharmaceutically acceptable carrier per dose. In some embodiments of the present disclosure, the amount of Mycobacterial whole cell lysate administered to the Sus is from about 100 to about 400 μg of Mycobacterial whole cell lysate per mL of a pharmaceutically acceptable carrier per dose.

In some embodiments of the present disclosure, the Mycobacterial whole cell lysate is contained in a multiple-dose vial prior to administration. The multiple-dose vial containing the Mycobacterial whole cell lysate of the present disclosure can be made of glass, plastic, or other material. In some embodiments, the multiple-dose vial includes from about 1 to about 1000 doses of the Mycobacterial whole cell lysate. In some embodiments, the multiple-dose vial includes from about 1 to about 500 doses of the Mycobacterial whole cell lysate. In some embodiments, the multiple-dose vial includes from about 1 to about 250 doses of the Mycobacterial whole cell lysate. In some embodiments, the multiple-dose vial includes from about 1 to about 100 doses of the Mycobacterial whole cell lysate. In some embodiments, the multiple-dose vial includes from about 1 to about 50 doses of the Mycobacterial whole cell lysate. In some embodiments, the multiple-dose vial includes from about 1 to about 25 doses of the Mycobacterial whole cell lysate.

In some embodiments of the present disclosure, the Mycobacterial whole cell lysate is administered as a multiple dose regimen. In some embodiments of the present disclosure, the multiple dose regimen is a time period of approximately 7 days. In some embodiments of the present disclosure, the multiple dose regimen is a time period of approximately 14 days. In some embodiments of the present disclosure, the multiple dose regimen is a time period of approximately one month. In some embodiments of the present disclosure, the multiple dose regimen is a time period of approximately two months. In some embodiments of the present disclosure, the multiple dose regimen is a time period of approximately three months. In some embodiments of the present disclosure, the multiple dose regimen is a time period of approximately four months. In some embodiments of the present disclosure, the multiple dose regimen is a time period of approximately five months. In some embodiments of the present disclosure, the multiple dose regimen is a time period of approximately six months.

In some embodiments of the present disclosure, the Mycobacterial whole cell lysate is administered as a single dose. In yet another embodiment of the present disclosure, the Mycobacterial whole cell lysate is administered as a single unit dose. As used herein, the term “unit dose” is a predetermined amount of Mycobacterial whole cell lysate. The amount of Mycobacterial whole cell lysate is generally equal to the dosage of Mycobacterial whole cell lysate that would be administered to a Sus or a convenient fraction of such a dosage such as, for example, one-half or one-third of such a dosage. According to the methods of the present disclosure, the terms “single dose” and “single unit dose” include embodiments wherein the composition can be administered as a single application and administered as multiple applications.

In some embodiments of the present disclosure, the Mycobacterial whole cell lysate is provided as a dry powder or granules which are reconstituted with water or other aqueous medium prior to first use. In some embodiments, the reconstitution with water or other aqueous medium forms an aqueous suspension. In some embodiments, the aqueous suspension is contained in a multiple-dose vial as described herein and has any number of doses of the Mycobacterial whole cell lysate as described herein. In some embodiments, the aqueous suspension is administered as a single dose as described herein. In some embodiments, the aqueous suspension is administered as a single unit dose as described herein. One of ordinary skill in the art understands that the present disclosure envisions utilizing dry powder or granules of any size, shape, volume, etc. The “powder in a bottle” process, as used in the pharmaceutical industry and understood by the skilled artisan, is contemplated by the present disclosure, including any variations thereof.

In some embodiments of the present disclosure, the volume of the Mycobacterial whole cell lysate administered to a Sus per dose varies. For example, the route of administration and device used to administer the Mycobacterial whole cell lysate can cause variations in the volume of the Mycobacterial whole cell lysate administered to a Sus per dose. In some embodiments of the present disclosure, the volume per dose is from about 0.001 to about 50 mL per dose. In some embodiments of the present disclosure, the volume per dose is from about 0.01 to about 25 mL per dose. In some embodiments of the present disclosure, the volume per dose is from about 0.1 to about 10 mL per dose. In some embodiments of the present disclosure, the volume per dose is from about 0.1 to about 5 mL per dose. In some embodiments of the present disclosure, the volume per dose is from about 1 to about 5 mL per dose. In some embodiments of the present disclosure, the volume per dose is from about 1 to about 2 mL per dose. In some embodiments of the present disclosure, the volume per dose is less than about 1 mL per dose.

The methods of the present disclosure utilize administration of a Mycobacterial whole cell lysate to a Sus to prime the Sus' immune system within an effective period of time after the Sus is born. As used herein, the term “effective period of time” means a time period sufficiently long enough to provide the desired administration to obtain the desired priming result. In some embodiments of the present disclosure, the Mycobacterial whole cell lysate is administered to the Sus from immediately after birth to about 1 hour of age. In some embodiments of the present disclosure, the Mycobacterial whole cell lysate is administered to the Sus from about 1 hour to about 24 hours of age. In some embodiments of the present disclosure, the Mycobacterial whole cell lysate is administered to the Sus from about 24 hours to about 1 week of age. In some embodiments of the present disclosure, the Mycobacterial whole cell lysate is administered to the Sus from about 1 week to about 1 month of age. In some embodiments of the present disclosure, the Mycobacterial whole cell lysate is administered to the Sus from about 1 month to about 2 months of age. In some embodiments of the present disclosure, the Mycobacterial whole cell lysate is administered to the Sus from about 2 months to about 3 months of age. In some embodiments of the present disclosure, the Mycobacterial whole cell lysate is administered to the Sus from about 3 months to about 4 months of age. In some embodiments of the present disclosure, the Mycobacterial whole cell lysate is administered to the Sus from about 4 months to about 8 months of age. In some embodiments of the present disclosure, the Mycobacterial whole cell lysate is administered to the Sus from about 8 months to about 12 months of age. In some embodiments of the present disclosure, the Mycobacterial whole cell lysate is administered to the Sus from about 12 months to about 24 months of age. In some embodiments of the present disclosure, the Mycobacterial whole cell lysate is administered to the Sus from about 24 months to about 36 months of age. In some embodiments of the present disclosure, the Mycobacterial whole cell lysate is administered to the Sus from about 36 months to about 48 months of age.

In some embodiments, the methods of the present disclosure can be intranasally administered to a Sus according to the doses shown in TABLE 1.

TABLE 1

| | | | | Dose | | |
|---|---|---|---|---|---|---|
| | | | | Vol- | | |
| Growth | | Weight | Micrograms | ume | | |
| Period | Weeks | (Kg) | (μg) | (mL) | μg/kg | μg/mL |
| Pre- | 1 to 3 | 2 to 6 | 50 to 250 | 1 | 8 to 125 | 50 to 250 |
| Starter | | | | | | |
| Starter | 4 to 6 | 5 to 12 | 100 to 500 | 2 | 8 to 100 | 50 to 250 |
| Grower | 7 to 10 | 10 to 26 | 150 to 750 | 3 | 6 to 75 | 50 to 250 |
| Develop | 11 to 16 | 25 to 58 | 200 to 1000 | 4 | 3 to 40 | 50 to 250 |
| Finisher | 17 to 22 | 55 to 100 | 250 to 1500 | 5 | 3 to 27 | 50 to 300 |
| Breeder | 22 to + | 100 to + | 250 to 1500 | 5 | 3 to 15 | 50 to 300 |

Weeks: Age of Sus in Weeks
Weight: Weight of Sus
μg: μg of Mycobacterial WCL
μg/kg: μg of Mycobacterial WCL per kg of Sus body weight
μg/mL: μg of Mycobacterial WCL per mL of a pharmaceutically acceptable carrier

The Mycobacterial whole cell lysate utilized in the methods of the present disclosure may optionally be combined with one or more pharmaceutically acceptable carriers. A non-limiting list of pharmaceutically acceptable carriers that can be utilized in the methods of the present disclosure include without limitation water or saline, gel, salve, solvent, oil, diluent, fluid ointment base, liposome, micelle, giant micelle, synthetic polymer, emulsion, a solid particle made of lipid, and the like. As the skilled artisan understands, any diluent known in the art may be utilized in accordance with the present disclosure. In some embodiments of the present disclosure, the diluent is water soluble. In some embodiments of the present disclosure, the diluent is water insoluble. As used herein, the term “diluent” includes without limitation water, saline, phosphate buffered saline (PBS), dextrose, glycerol, ethanol, buffered sodium or ammonium acetate solution, or the like and combinations thereof.

The following embodiments are also contemplated:

1. A method of priming a Sus' immune system, the method comprising administering an effective amount of a Mycobacterial whole cell lysate to the Sus within an effective period of time after the Sus is born.

2. The method of clause 1, wherein the Mycobacterial whole cell lysate is prepared from Mycobacterium smegmatis.

3. The method of clause 1 or clause 2, wherein the Mycobacterial whole cell lysate has not undergone purification.

4. The method of any one of clauses 1 to 3, wherein the Mycobacterial whole cell lysate is unfractionated.

5. The method of any one of clauses 1 to 4, wherein the Mycobacterial whole cell lysate is not delipidated.

6. The method of any one of clauses 1 to 5, wherein the Mycobacterial whole cell lysate is not deproteinized.

7. The method of any one of clauses 1 to 6, wherein the administration is selected from the group consisting of oral, intravenous, subcutaneous, intramuscular, intraperitoneal, intradermal, intraocular, intrapulmonary, intranasal, transdermal, subdermal, topical, mucosal, nasal, impression into skin, intravaginal, intrauterine, intracervical, and rectal.

8. The method of any one of clauses 1 to 7, wherein the administration is mucosal.

9. The method of any one of clauses 1 to 8, wherein the administration is intranasal.

10. The method of any one of clauses 1 to 9, wherein the amount of Mycobacterial whole cell lysate administered to the Sus is from about 0.0001 to about 1000 μg of Mycobacterial whole cell lysate per mL of a pharmaceutically acceptable carrier per dose.

11. The method of any one of clauses 1 to 10, wherein the amount of Mycobacterial whole cell lysate administered to the Sus is from about 50 to about 500 μg of Mycobacterial whole cell lysate per mL of a pharmaceutically acceptable carrier per dose.

12. The method of any one of clauses 1 to 11, wherein the amount of Mycobacterial whole cell lysate administered to the Sus is from about 100 to about 400 μg of Mycobacterial whole cell lysate per mL of a pharmaceutically acceptable carrier per dose.

13. The method of any one of clauses 1 to 12, wherein the Mycobacterial whole cell lysate is administered as a single dose.

14. The method of any one of clauses 1 to 13, wherein the Mycobacterial whole cell lysate is administered as a single unit dose.

15. The method of any one of clauses 1 to 12, wherein the Mycobacterial whole cell lysate is administered as a multiple dose regimen.

16. The method of any one of clauses 1 to 9, wherein the volume per dose is from about 0.001 to about 50 mL per dose.

17. The method of any one of clauses 1 to 9, wherein the volume per dose is from about 0.01 to about 25 mL per dose.

18. The method of any one of clauses 1 to 9, wherein the volume per dose is from about 0.1 to about 10 mL per dose.

19. The method of any one of clauses 1 to 9, wherein the volume per dose is from about 1 to about 5 mL per dose.

20. The method of any one of clauses 1 to 9, wherein the volume per dose is from about 1 to about 2 mL per dose.

21. The method of any one of clauses 1 to 20, wherein the Mycobacterial whole cell lysate is administered to the Sus from immediately after birth to about 1 hour of age.

22. The method of any one of clauses 1 to 20, wherein the Mycobacterial whole cell lysate is administered to the Sus from about 1 hour to about 24 hours of age.

23. The method of any one of clauses 1 to 20, wherein the Mycobacterial whole cell lysate is administered to the Sus from about 24 hours to about 1 week of age.

24. The method of any one of clauses 1 to 20, wherein the Mycobacterial whole cell lysate is administered to the Sus from about 1 week to about 1 month of age.

25. The method of any of clauses 1 to 20, wherein the Mycobacterial whole cell lysate is administered to the Sus from about 1 month to about 2 months of age.

26. The method of any one of clauses 1 to 20, wherein the Mycobacterial whole cell lysate is administered to the Sus from about 2 months to about 3 months of age.

27. The method of any of clauses 1 to 20, wherein the Mycobacterial whole cell lysate is administered to the Sus from about 3 months to about 4 months of age.

28. The method of any one of clauses 1 to 27, wherein priming a Sus' immune system comprises priming white blood cells.

29. The method of any one of clauses 1 to 27, wherein priming a Sus' immune system comprises priming T cells.

30. The method of any one of clauses 1 to 27, wherein priming a Sus' immune system comprises priming monocytes.

31. The method of any one of clauses 1 to 27, wherein priming a Sus' immune system comprises priming macrophages.

32. The method of any one of clauses 1 to 27, wherein priming a Sus' immune system comprises priming alveolar macrophages.

33. The method of any one of clauses 1 to 28, wherein the primed white blood cells exhibit enhanced production of interferon gamma in response to a stimulus.

34. The method of any one of clauses 1 to 9, wherein the Mycobacterial whole cell lysate is combined with a pharmaceutically acceptable carrier.

35. The method of any one of clauses 1 to 34, wherein the Sus is a pig.

36. A Mycobacterial whole cell lysate for use in priming a Sus' immune system comprising administering an effective amount of the Mycobacterial whole cell lysate to the Sus within an effective period of time after the Sus is born.

37. A Mycobacterial whole cell lysate for use according to clause 36, wherein the Mycobacterial whole cell lysate is prepared from Mycobacterium smegmatis.

38. A Mycobacterial whole cell lysate for use according to clause 36 or clause 37, wherein the Mycobacterial whole cell lysate has not undergone purification.

39. A Mycobacterial whole cell lysate for use according to any one of clauses 36 to 38, wherein the Mycobacterial whole cell lysate is unfractionated.

40. A Mycobacterial whole cell lysate for use according to any one of clauses 36 to 39, wherein the Mycobacterial whole cell lysate is not delipidated.

41. A Mycobacterial whole cell lysate for use according to any one of clauses 36 to 40, wherein the Mycobacterial whole cell lysate is not deproteinized.

42. A Mycobacterial whole cell lysate for use according to any one of clauses 36 to 41, wherein the administration is selected from the group consisting of oral, intravenous, subcutaneous, intramuscular, intraperitoneal, intradermal, intraocular, intrapulmonary, transdermal, subdermal, topical, mucosal, nasal, and impression into skin.

43. A Mycobacterial whole cell lysate for use according to any one of clauses 36 to 42, wherein the administration is mucosal.

44. A Mycobacterial whole cell lysate for use according to any one of clauses 36 to 43, wherein the administration is intranasal.

45. A Mycobacterial whole cell lysate for use according to any one of clauses 36 to 44, wherein the amount of Mycobacterial whole cell lysate administered to the Sus is from about 0.0001 to about 1000 μg of Mycobacterial whole cell lysate per mL of a pharmaceutically acceptable carrier per dose.

46. A Mycobacterial whole cell lysate for use according to any one of clauses 36 to 45, wherein the amount of Mycobacterial whole cell lysate administered to the Sus is from about 50 to about 500 μg of Mycobacterial whole cell lysate per mL of a pharmaceutically acceptable carrier per dose.

47. A Mycobacterial whole cell lysate for use according to any one of clauses 36 to 46, wherein the amount of Mycobacterial whole cell lysate administered to the Sus is from about 100 to about 400 μg of Mycobacterial whole cell lysate per mL of a pharmaceutically acceptable carrier per dose.

48. A Mycobacterial whole cell lysate for use according to any one of clauses 36 to 47, wherein the Mycobacterial whole cell lysate is administered as a single dose.

49. A Mycobacterial whole cell lysate for use according to any one of clauses 36 to 48, wherein the Mycobacterial whole cell lysate is administered as a single unit dose.

50. A Mycobacterial whole cell lysate for use according to any one of clauses 36 to 47, wherein the Mycobacterial whole cell lysate is administered as a multiple dose regimen.

51. A Mycobacterial whole cell lysate for use according to any one of clauses 36 to 44, wherein the volume per dose is from about 0.001 to about 50 mL per dose.

52. A Mycobacterial whole cell lysate for use according to any one of clauses 36 to 44, wherein the volume per dose is from about 0.01 to about 25 mL per dose.

53. A Mycobacterial whole cell lysate for use according to any one of clauses 36 to 44, wherein the volume per dose is from about 0.1 to about 10 mL per dose.

54. A Mycobacterial whole cell lysate for use according to any one of clauses 36 to 44, wherein the volume per dose is from about 1 to about 5 mL per dose.

55. A Mycobacterial whole cell lysate for use according to any one of clauses 36 to 44, wherein the volume per dose is from about 1 to about 2 mL per dose.

56. A Mycobacterial whole cell lysate for use according to any one of clauses 36 to 55, wherein the Mycobacterial whole cell lysate is administered to the Sus from immediately after birth to about 1 hour of age.

57. A Mycobacterial whole cell lysate for use according to any one of clauses 36 to 55, wherein the Mycobacterial whole cell lysate is administered to the Sus from about 1 hour to about 24 hours of age.

58. A Mycobacterial whole cell lysate for use according to any one of clauses 36 to 55, wherein the Mycobacterial whole cell lysate is administered to the Sus from about 24 hours to about 1 week of age.

59. A Mycobacterial whole cell lysate for use according to any one of clauses 36 to 55, wherein the Mycobacterial whole cell lysate is administered to the Sus from about 1 week to about 1 month of age.

60. A Mycobacterial whole cell lysate for use according to any one of clauses 36 to 55, wherein the Mycobacterial whole cell lysate is administered to the Sus from about 1 month to about 2 months of age.

61. A Mycobacterial whole cell lysate for use according to any one of clauses 36 to 55, wherein the Mycobacterial whole cell lysate is administered to the Sus from about 2 months to about 3 months of age.

62. A Mycobacterial whole cell lysate for use according to any one of clauses 36 to 55, wherein the Mycobacterial whole cell lysate is administered to the Sus from about 3 months to about 4 months of age.

63. A Mycobacterial whole cell lysate for use according to any one of clauses 36 to 62, wherein priming a Sus' immune system comprises priming white blood cells.

64. A Mycobacterial whole cell lysate for use according to any one of clauses 36 to 62, wherein priming a Sus' immune system comprises priming T cells.

65. A Mycobacterial whole cell lysate for use according to any one of clauses 36 to 62, wherein priming a Sus' immune system comprises priming monocytes.

66. A Mycobacterial whole cell lysate for use according to any one of clauses 36 to 62, wherein priming a Sus' immune system comprises priming macrophages. 67. A Mycobacterial whole cell lysate for use according to any one of clauses 36 to 62, wherein priming a Sus' immune system comprises priming alveolar macrophages.

68. A Mycobacterial whole cell lysate for use according to any one of clauses 36 to 63, wherein the primed white blood cells exhibit enhanced production of interferon gamma in response to a stimulus. 69. A Mycobacterial whole cell lysate for use according to any one of clauses 36 to 44, wherein the Mycobacterial whole cell lysate is combined with a pharmaceutically acceptable carrier.

70. A Mycobacterial whole cell lysate for use according to any one of clauses 36 to 69, wherein the Sus is a pig. 71. The use of a Mycobacterial whole cell lysate for the manufacture of a medicament for use in priming a Sus' immune system comprising administering an effective amount of the Mycobacterial whole cell lysate to the Sus within an effective period of time after the Sus is born.

72. The use of clause 71, wherein the Mycobacterial whole cell lysate is prepared from Mycobacterium smegmatis.

73. The use of clause 71 or clause 72, wherein the Mycobacterial whole cell lysate has not undergone purification.

74. The use of any one of clauses 71 to 73, wherein the Mycobacterial whole cell lysate is unfractionated.

75. The use of any one of clauses 71 to 74, wherein the Mycobacterial whole cell lysate is not delipidated.

76. The use of any one of clauses 71 to 75, wherein the Mycobacterial whole cell lysate is not deproteinized.

77. The use of any one of clauses 71 to 76, wherein the administration is selected from the group consisting of oral, intravenous, subcutaneous, intramuscular, intraperitoneal, intradermal, intraocular, intrapulmonary, transdermal, subdermal, topical, mucosal, nasal, and impression into skin.

78. The use of any one of clauses 71 to 77, wherein the administration is mucosal.

79. The use of any one of clauses 71 to 78, wherein the administration is intranasal.

80. The use of any one of clauses 71 to 79, wherein the amount of Mycobacterial whole cell lysate administered to the Sus is from about 0.0001 to about 1000 μg of Mycobacterial whole cell lysate per mL of a pharmaceutically acceptable carrier per dose.

81. The use of any one of clauses 71 to 80, wherein the amount of Mycobacterial whole cell lysate administered to the Sus is from about 50 to about 500 μg of Mycobacterial whole cell lysate per mL of a pharmaceutically acceptable carrier per dose.

82. The use of any one of clauses 71 to 81, wherein the amount of Mycobacterial whole cell lysate administered to the Sus is from about 100 to about 400 μg of Mycobacterial whole cell lysate per mL of a pharmaceutically acceptable carrier per dose.

83. The use of any one of clauses 71 to 82, wherein the Mycobacterial whole cell lysate is administered as a single dose.

84. The use of any one of clauses 71 to 83, wherein the Mycobacterial whole cell lysate is administered as a single unit dose.

85. The use of any one of clauses 71 to 82, wherein the Mycobacterial whole cell lysate is administered as a multiple dose regimen.

86. The use of any one of clauses 71 to 79, wherein the volume per dose is from about 0.001 to about 50 mL per dose.

87. The use of any one of clauses 71 to 79, wherein the volume per dose is from about 0.01 to about 25 mL per dose.

88. The use of any one of clauses 71 to 79, wherein the volume per dose is from about 0.1 to about 10 mL per dose.

89. The use of any one of clauses 71 to 79, wherein the volume per dose is from about 1 to about 5 mL per dose.

90. The use of any one of clauses 71 to 79, wherein the volume per dose is from about 1 to about 2 mL per dose.

91. The use of any one of clauses 71 to 90, wherein the Mycobacterial whole cell lysate is administered to the Sus from immediately after birth to about 1 hour of age.

92. The use of any one of clauses 71 to 90, wherein the Mycobacterial whole cell lysate is administered to the Sus from about 1 hour to about 24 hours of age.

93. The use of any one of clauses 71 to 90, wherein the Mycobacterial whole cell lysate is administered to the Sus from about 24 hours to about 1 week of age.

94. The use of any one of clauses 71 to 90, wherein the Mycobacterial whole cell lysate is administered to the Sus from about 1 week to about 1 month of age.

95. The use of any of clauses 71 to 90, wherein the Mycobacterial whole cell lysate is administered to the Sus from about 1 month to about 2 months of age.

96. The use of any one of clauses 71 to 90, wherein the Mycobacterial whole cell lysate is administered to the Sus from about 2 months to about 3 months of age.

97. The use of any of clauses 71 to 90, wherein the Mycobacterial whole cell lysate is administered to the Sus from about 3 months to about 4 months of age.

98. The use of any one of clauses 71 to 97, wherein priming a Sus' immune system comprises priming white blood cells.

99. The use of any one of clauses 71 to 97, wherein priming a Sus' immune system comprises priming T cells.

100. The use of any one of clauses 71 to 97, wherein priming a Sus' immune system comprises priming monocytes.

101. The use of any one of clauses 71 to 97, wherein priming a Sus' immune system comprises priming macrophages.

102. The use of any one of clauses 71 to 97, wherein priming a Sus' immune system comprises priming alveolar macrophages.

103. The use of any one of clauses 71 to 98, wherein the primed white blood cells exhibit enhanced production of interferon gamma in response to a stimulus.

104. The use of any one of clauses 71 to 79, wherein the Mycobacterial whole cell lysate is combined with a pharmaceutically acceptable carrier.

105. The use of any one of clauses 71 to 104, wherein the Sus is a pig.

An example of a Mycobacterial whole cell lysate and process of making the Mycobacterial whole cell lysate is provided. A seed stock is created by growing Mycobacterium smegmatis strain designation mc²155 (first generation) in Middlebrook7H9 Broth and OADC (7H9+OADC) medium to generate 50 to 100 seed stocks for further processing such as storing frozen seed stocks for future inoculations of culture media. A commercially available source of Mycobacterium smegmatis mc²155 is Mycobacterium smegmatis (Trevisan) Lehmann and Neumann (ATCC® 700084™). A commercially available source of Middlebrook7H9 Broth suitable for the present disclosure is BD (Becton, Dickinson, and Company) Difco™ Middlebrook7H9 Broth.

As one of ordinary skill in the art knows, OADC is the abbreviation for oleic acid, albumin, dextrose, and catalase, which is used in media for Mycobacterial species. The OADC complement includes the components in the amounts identified in TABLE 2.

TABLE 2

| Components | 0.25 L | 0.5 L | 0.75 L | 1 L |
|---|---|---|---|---|
| Water (mL) | 237.5 | 475 | 712.5 | 950 |
| NaCl (g) | 2.025 | 4.05 | 6.075 | 8.1 |
| BSA (g) | 12.5 | 25 | 37.5 | 50 |
| D-glucose (g) | 5 | 10 | 15 | 20 |
| Sodium Oleate | 7.5 | 15 | 22.5 | 30 |
| (mL) | | | | |

The OADC complement is prepared by first dissolving NaCl in water in an appropriately sized container based on the amounts of the components provided in TABLE 2. BSA is slowly added and the combination is stirred until the BSA is dissolved, which can take up to an hour. D-isomer glucose (“D-glucose”) is added to the combination. The pH of the combination is adjusted to 7 by adding suitable amounts of NaOH. In a second container, sodium oleate is prepared, and its components include 240 mL of water, 4.8 mL of 6MNaOH, and 4.8 mL of oleic acid. The components are warmed to 56° C. and swirled until the components become a clear solution. The sodium oleate solution is added to the OADC complement. In a hood, the combination is filtered into a sterile bottle. The bottle is covered with aluminum foil and stored at 4° C.

The 7H9+OADC Media is prepared by using the components in the amounts shown in TABLE 3. The 7H9 Media is prepared by first adding glycerol, media, and water to an autoclaved Erlenmeyer and mixing the components. The OADC complement is added to the combination, and the combination is mixed. In a hood, the media is filtered into a sterile bottle. The bottle is covered with aluminum foil and stored at 4° C.

TABLE 3

| Components | 0.5 L | 1 L | 1.5 L | 2 L | 2.5 L | 3 L |
|---|---|---|---|---|---|---|
| 7H9 (g) | 2.35 | 4.7 | 7.05 | 9.4 | 11.75 | 14.1 |
| OADC (mL) | 50 | 100 | 150 | 200 | 250 | 300 |
| Water (mL) | 450 | 900 | 1350 | 1800 | 2250 | 2700 |
| Glycerol (mL) | 1 | 2 | 3 | 4 | 5 | 6 |

A culture of Mycobacterium smegmatis mc²155 can be grown on 7H9+OADC Media or GAS Media using a first generation stock of Mycobacterium smegmatis mc²155. The first step is starting the culture by preparing growth medium to be used (7H9 or GAS) and aliquot into tubes at 10 mL to 50 mL. The starting culture is inoculated by rapidly thawing the Mycobacterium smegmatis seed culture and aseptically transferring 1 mL frozen stock to 10 mL culture media. The culture tubes are incubated at 37° C. for 24 to 72 hours until Mycobacterial growth is evident and robust.

The next step is sub-culturing. After starting from frozen stock, cultures are expanded by removing the growing Mycobacterial culture and adding to fresh growth medium at 10% of the total final volume. For example, 10 mL of growing seed will be added to 100 mL of new media. The newly inoculated cultures are returned to incubation at 37° C. for 24 to 72 hours.

The next step is the final culture. Once achieving the final total volume of Mycobacterial culture following the sub-culture method, the production fermentation vessel is incubated post-inoculation for 72 hours at 37° C. with aeration and mixing.

The next step is harvesting. Culture media containing the Mycobacteria is removed from the fermentation vessel and centrifuged to pellet the cells at 3,000 rpm (2,000×g) for 15 minutes. Alternatively, the culture can sit undisturbed for 10 to 15 minutes allowing the heavier Mycobacteria to settle to the bottom of the collection vessel. The supernatant fluids are removed either by pouring off the liquid or aspirating the liquid from above the settled/centrifuged pellet. Phosphate buffered saline is added to the settled/centrifuged pellet to wash the Mycobacteria. PBS is removed again by settling/centrifugation and the pellet washed a total of 3 times before freezing/lysing.

The next step is freezing/lysing. The collected and washed pellet may be frozen until processed further or the pellet can be processed immediately without freezing. The pellet is suspended in lysis buffer (PBS with 8 mM EDTA), proteinase inhibitor, 250 ug/mL Dnase and 250 ug/mL Rnase to contain 2 grams (wet weight) Mycobacteria per mL of lysis buffer. Mycobacterial cells are broken using physical shear forces such as sonication, high pressure homogenization or lab scale homogenization with zirconia beads. Cell preparation is added to an equal volume of zirconia/silica beads (0.1 mM) and mix for up to 30 minutes.

The next step is clarification. After lysis of the Mycobacterial cells, the material is clarified again by allowing the larger beads and unbroken cell components to settle in a container. Centrifugation may also be used to speed up sedimentation. After clarified, the resulting material is filtered through a 0.22 micron filter and stored in aliquots frozen at −20° C. or less.

The last step is analytical testing. The final frozen material is tested for endotoxin, TNF-alpha stimulating capability, total protein, and sterility.

The present disclosure is further described by the following non-limiting Examples. Alveolar Macrophages (AMΦ) are the main type of innate immune system cells maintaining immune homeostasis in the airways. Without being bound by any theory, the pro-inflammatory milieu created by AMΦ responding to microbial products is thought to be crucial in development of the adaptive immune response against respiratory virus infection. To test the ability of the Mycobacterium smegmatis WCL to stimulate a pro-inflammatory response in AMΦ, studies can be conducted to measure the tumor necrosis factor (TNF)-alpha (“TNF-α”) response of porcine AMΦ to Mycobacterium smegmatis WCL exposure. A representative sampling of this type of cell consists of the porcine AMΦ ZMAC cell.

TNF alpha is primarily a macrophage-derived cytokine. It induces the signal transduction, activation, and translocation of NF-κB which acts as the “master switch” for transactivation of a number of cytokine genes involved in mediating innate host defense. Along with other pro-inflammatory cytokines such as INF gamma and IL-12, TNF alpha is involved in activation of macrophages and neutrophils, augmentation of professional phagocyte-dependent functions, and direction of cell-mediated immunity.

Porcine Alveolar Macrophages

The porcine AMΦ cell line, ZMAC-4, can be derived from the lungs of porcine fetuses and consists of phagocytic cells that express several surface markers characteristic of AMΦ, including CD14, CD45, CD163, and CD172. ZMAC cells have been shown to efficiently support the growth of PRRSV. ZMAC cells can be cultured in RPMI-1640 Medium containing 1-glutamine (which is commercially available from a number of sources including Mediatech, Herndon, Va., USA) and supplemented with 10% fetal bovine serum (FBS) (GIBCO®, which is commercially available from Thermo Fisher Scientific, Waltham, Mass., USA), 1 mM sodium pyruvate, and 1×non-essential amino acids (which is commercially available from Mediatech, among other sources) and kept at 37° C. in a 5% CO₂ atmosphere. Maintenance of ZMAC cells also requires the inclusion of 10 nanograms per milliliters (ng/mL) recombinant Mouse Macrophage Colony Stimulating Factor (“M-CSF”) (which is commercially available from Shenandoah Biotechnology, Inc.™, Warwick, Pa., USA).

Stimulation of Porcine AMΦ

ZMAC cells are cultured at 5×10\hat{ }5 cells per milliliters (cells/mL) in each individual well of 48-wells plate (Corning®, New York, USA) and are subsequently exposed to either mock medium, 100 ng/mL lipopolysaccharide (“LPS”) or Lipoarabinomannan from Mycobacterium smegmatis (LAM-MS; InvivoGen, San Diego, Calif.) at either 5, 1.67 or 0.56 mcg/mL or a crude Mycobacterium smegmatis WCL at either 10, 5, 1.67 or 0.56 mcg/mL are cultured for either 6 12 or 24 hours. At one of these time points, culture supernatants are harvested and stored at −20° C. until testing.

Quantitation of TNF-α

The medium used to culture porcine alveolar macrophages that had been mock treated or treated with LPS, purified LAM or crude Mycobacterium smegmatis WCL are assayed for the presence of TNF-alpha by using a specific enzyme-linked immunosorbent assay (“ELISA”). For the detection of TNF-α, individual wells of a Nunc Immulon 4HBX 96-well plate (Thermo Fisher Scientific) that had been coated for 16 hours at 4° C. with 50 microliters (μl) of 32 micrograms per microliters (m/mL) Porcine TNF-alpha MAb (Clone 103304, which is commercially available from R&D systems, Minneapolis, Minn., USA) in 0.1 M carbonate buffer (pH 9.6) are washed 3 times with PBS containing 0.05% Tween 20 (PBS-T) and incubated with blocking solution (1% BSA in PBS-T) for 1 hour at RT. After three washes with PBS-T, 50 μl culture supernatants and TNF-α standard (R&D systems) diluted in RPMI complete medium are added to duplicate wells and left for 2 hour at RT. After washing 5 times with PBS-T, each well is incubated with 50 μl of PBS-T containing 2.5 μg/mL biotin-labeled, Porcine TNF-alpha MAb (Clone 103302, which is commercially available from R&D systems) and 0.5% BSA blocking solution at RT for 1.5 hours. After 5 washes with PBS-T, each well is incubated with 50 μl PBS-T containing 20 ng/mL HRP-Conjugated Streptavidin, which is commercially available from Thermo Fisher Scientific, for 20 min at RT and then again washed 5 times with PBS-T. Color development is initiated at RT with the addition of 100 μl TMB substrate (which is commercially available from KPL, Gaithersburg, Md., US) per well and terminated with 100 μl 1 M phosphoric acid. Optical densities are determined at 450 nm with a SpectraMax® Plus Microplate Reader (which is commercially available from Molecular Devices, Sunnyvale, Calif., USA). Results are averaged and the amounts of TNF-α are determined by comparison to a standard curve generated from the values obtained with known quantities of TNF-α.

### Example 1

The Significant Production of TNF-Alpha by the Porcine AMΦ Stimulated with Mycobacterium smematis WCL

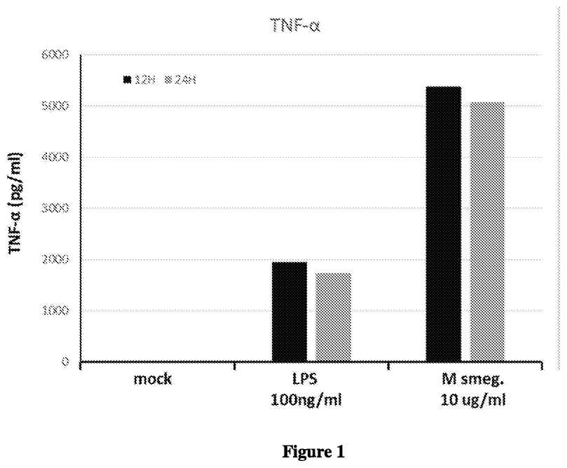

The capability of ZMAC cells to produce TNF-alpha in response to LPS had been demonstrated in previous studies. To test the immune-stimulating activity of Mycobacterium smegmatis WCL, ZMAC cells are exposed to Mycobacterium smegmatis WCL grown in 7H9 broth, which is optimized for Mycobacteria culture. A high concentration of 10 ug/mL of Mycobacterium smegmatis is initially used to stimulate the cells for 12 and 24 hours. As illustrated in FIG. 1, the results show a burst in the production of tumor necrosis factor (TNF)-alpha when porcine alveolar macrophages (AMΦ) ZMAC are exposed to Mycobacterium smegmatis WCL compared to the lower production of TNF-alpha when porcine AMΦ ZMAC are exposed to the bacterial product lipopolysaccharide (LPS), which is a potent stimulant of TNF-alpha production.

### Example 2

Most of the Production of TNF-Alpha by AMΦ in Response to Mycobacterium smegmatis WCL Occurs in the First 6 Hours after Stimulation

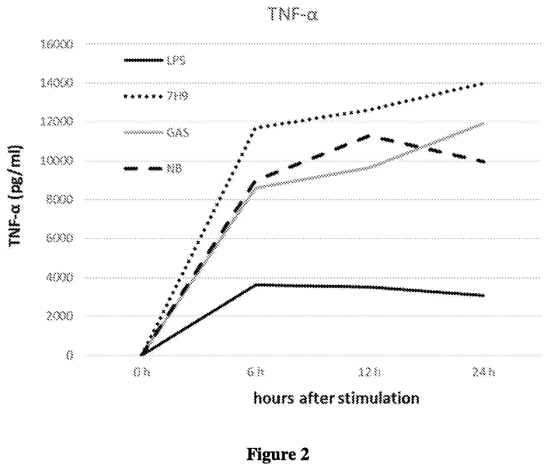

While the data from previous experiments show that a significant amount of TNF-alpha is produced by 12 hours after stimulation, it appears that there is no further production of this cytokine during the time period of 12 hours to 24 hours. This observation led the inventors of the present disclosure to question whether the TNF-alpha response to Mycobacterium smegmatis WCL resembles the similar expression kinetics that have been observed for this cytokine in response to LPS stimulation, which usually peaks within 4-6 hours after stimulation. Therefore, a temporal analysis is set up to establish TNF-alpha production kinetics in response to stimulation of Mycobacterium smegmatis WCL. In this experiment, the inventors of the present disclosure also include two other WCLs prepared from the same Mycobacterium smegmatis but cultured in different broth types. As illustrated in FIG. 2, the results from this temporal analysis demonstrate that the majority of TNF-alpha expression activity occurs within 6 hours after stimulation, and this expression kinetic is similar in response to all three Mycobacterium smegmatis WCL preparations tested. The similar kinetics and intensity of the TNF-alpha response of macrophages to the three different preparations of WCL is similar and these results suggest that all three preparations have similar compositions with regards to their ability to stimulate macrophages to produce TNF-alpha.

### Example 3

The Culture Media Used to Grow Mycobacterium smegmatis Affects the TNF-Alpha Induction Capability of the WCL

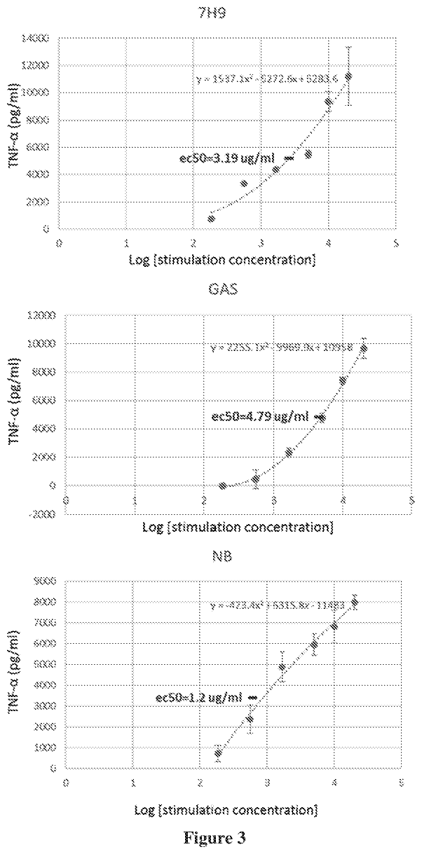

Despite the expression kinetic being independent of the growth condition of Mycobacterium smegmatis, it is noticed that the maximal TNF-alpha response differs from the lysate preparations as demonstrated in FIG. 2. These results suggest that the culture media used to grow the Mycobacterium smegmatis might affect the potency of the WCL in inducing the TNF-alpha response of AMΦ cells. To test this theory, a dose-response curve for each WCL is established in a potency analysis. As shown in FIG. 3, while the results indicate that all three lysates are capable of inducing a good TNF-alpha response of AMΦ, the interpretation of the potency is complicated by the slight differences in the total amount of TNF-alpha produced. In this study, for example, FIG. 3 shows that the lysate prepared from Mycobacterium smegmatis grown in NB broth yielded the lowest half-effective dose (ec50=1.2 ug/mL) suggesting the greater potency of this lysate. However, it can be appreciated that the maximum response by that lysate is about 80% of those responses induced by the WCL prepared from Mycobacterium smegmatis grown in 7H9 or GAS broth.

The complicated interpretation of potency is removed by excluding lysates prepared from NB broth. From this analysis it is reasonable to conclude that the lysate prepared from Mycobacterium smegmatis grown in 7H9 medium is slightly more potent than that prepared from bacteria grown in GAS medium. Accordingly, as illustrated in FIG. 3, this study demonstrates that the potency (as indicated by the 50%-effective dose) of the Mycobacterium smegmatis WCL extract can be affected by the type of growth media that is used to culture the Mycobacterium smegmatis in order to prepare the bacterial cell mass to prepare the WCL. Of the three different media tested, the GAS medium appeared to be the best with a 50%-effective dose of 4.79 mcg/mL, followed by the 7H9 medium, with a 50%-effective dose of 3.19 mcg/mL media, and followed by the NB media with a 50%-effective dose of 1.2 mcg/mL.

### Example 4

Mycobacterium smegmatis WCL Induces a Significantly Greater TNF-Alpha Response than Purified Mycobacterial Cell Wall Component LAM-MS

Several components of a Mycobacteria cell wall are known to have immune stimulatory activity including, for example, muramyl dipeptide (MDP), trehalose dimycolate (TDM), and Mycobacteria cell wall Lipoarabinomannan (“LAM”). Mycobacteria-derived LAM, which is expressed by all Mycobacteria species, is known to activate macrophages by engaging the toll like receptor (TLR)-2 present in Mycobacteria cells.

LAM is the most characterized Mycobacteria call-wall component known to induce pro-inflammatory cytokine production including TNF-alpha via TLR2 pathway. This study compares the TNF-alpha response of ZMAC cells in response to stimulation with Mycobacterium smegmatis WCL relative to stimulation with LAM that was purified from Mycobacterium smegmatis (“LAM-MS”).

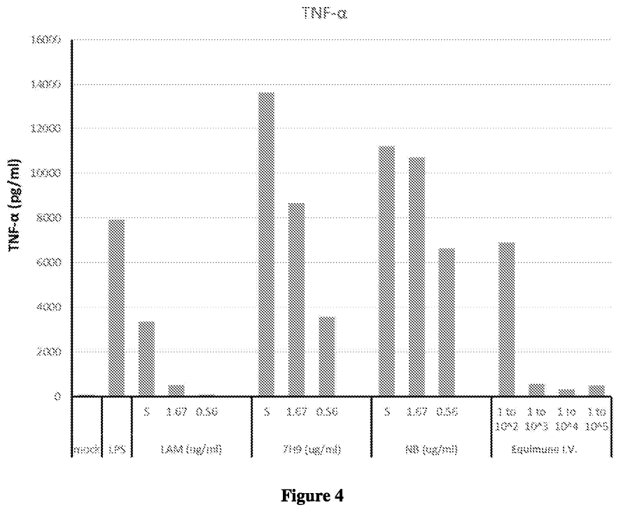

At the same stimulation concentration of the Mycobacterium smegmatis WCL and mycobacterial LAM-MS, the observed amount of TNF-alpha produced by ZMAC cells in response to stimulation with Mycobacterium smegmatis WCL is about four-fold higher than the amount of TNF-alpha produced by the same cells in response to stimulation with the commercially available LAM-MS (InVivoGen), which is illustrated in FIG. 4. Because the LAM of the Mycobacterium smegmatis is only a fraction of the Mycobacterium smegmatis WCL, these results suggest that other components of Mycobacterium smegmatis in addition to LAM are likely contributing to TNF-alpha production by an additive and possibly synergistic mechanism between the complex mixture of components present in the Mycobacterium smegmatis WCL.

### Example 5

Mycobacterium smegmatis WCL Induces a Significantly Greater TNF-Alpha Response Compared to Deproteinized and Delipidated Mycobacterial Cell Wall Extract (MCWE)

The TNF-alpha response of ZMAC cells in response to stimulation with Mycobacterium smegmatis WCL is compared to stimulation with Equimune I.V., a commercial product available from Bioniche Animal Health USA, Inc. (Athens, Ga.) having U.S. Veterinary License No. 289. The Equimune I.V. product is also encompassed by expired U.S. Pat. No. 4,744,984. Equimune I.V. is an emulsion of purified mycobacterium cell walls that have been extracted from Mycobacterium phlei. Since no concentration of the cell wall extract is indicated in the commercial product, a series of dilutions are tested for their ability to stimulate TNF-alpha production by ZMAC cells. The results of this experiment are illustrated in FIG. 4. Although a direct comparison for potency of the Equimune I.V. to the Mycobacterium smegmatis WCL is not possible since the amount of bacterial extract in the Equimune I.V. product is unknown, it is apparent that as little as 1.67 μg/mL of the Mycobacterium smegmatis WCL stimulated a stronger TNF-alpha response than Equimune I.V. Therefore, these results indicate that administering a Mycobacterial whole cell lysate, which includes the structural components of the Mycobacterial envelope, has an additive and possibly synergistic effect on the ZMAC cells compared to administering Equimune I.V., which likely has only cell wall components.

### Example 6

Administration of Mycobacterium smegmatis WCL to Pigs Results in Stimulation of the Pig's Immune System

A proof of concept study is conducted to evaluate the immunological effect of the Mycobacterium smegmatis WCL on swine. The Basic Study Design is shown in TABLE 4.

TABLE 4

| Animal Species | Swine (i.e., pig) | |
|---|---|---|
| Number of Animals | Sixteen (16) | |
| Age of Animal | Between Three (3) Weeks Old and Up to 14 | |
| | Days Post-Weaning and Acclimation | |
| Test Groups | 1) | Eight (8) - Placebo Controls (PBS); and |
| | 2) | Eight (8) - 500 μg of Mycobacterium |
| | | smegmatis WCL |
| Product Grouping | A) | Placebo (PBS); and |
| | B) | 500 μg of Mycobacterium smegmatis |
| | | WCL per mL of PBS |

| | |
|---|---|
| Product Administration | At Day 0, a Single, 1 mL Dose is Administered |
| | to all Swine via Intranasal Route Using a |
| | Neogen Corporation Prima Tech Nasal Sprayer |
| | having (1) a Syringe Prima Mist Vaccinator |
| | 2 mL (Part # 370334), (2) a Prima Mist |
| | Replacement Foam (Part # 364876), and (3) an |
| | applicator tip (Part # 3333) |

Basic Design Protocol

16 pigs are randomly assigned to 2 groups of 8 pigs and identified with ear tags. Pigs are either comingled together in one pen or not more than 2 pens located in the same production facility. Pigs are treated as described in TABLE 4. Pigs are housed in a production facility throughout the duration of the study. All pig work is conducted at the production facility. Pigs are returned to their herd for routine finishing and processed normally upon completion of the study. Blood is collected in the morning on the day of treatment at between 12-18 hours post-treatment and 3 days post-treatment.

The Proposed Planning is as follows. At Day 0 morning, pigs are randomly selected for study, tag, and bleed. Blood is sent to Aptimmune Biologics, Inc. in Champaign, Ill. immediately after collection. Blood is stored at ambient temperature. At Day 0 afternoon, ear tag numbers are sent to Aptimmune Biologics, Inc. for random assignment of pigs to study groups A and B. If two pens are being used, pigs are divided randomly between two pens. At Day 0 afternoon, administer treatments A and B to 8 pigs each per group assignment (target finish of treatment between 3 and 5 PM). At Day 1 early morning, all pigs are bled as early in the day as possible, targeting 12-18 hours post-treatment. All blood samples are sent to Aptimmune Biologics, Inc. and are stored at ambient temperature. At Day 3 morning, all pigs are bled in the morning and blood is sent to Aptimmune Biologics, Inc. as soon as possible at ambient temperature.

Analytical Testing protocol

1×10 mL sample of whole blood collected into Heparin containing tubes to prevent clotting is collected from each pig and identified with the ear tag number and date. Heparin treated blood is collected and analyzed for (1) TNF-alpha stimulation; (2) Natural Killer Subpopulation; and (3) B-Cell Subpopulation. Testing samples are delivered to Aptimmune Biologics, Inc. and processed immediately.

Schedule of Activities

At Day 0 morning, 16 pigs are tagged with unique numbered tags to enroll in study. Unhealthy animals are not used. While tagging animals, a 10 mL blood sample is collected into Heparin tubes (green top). Each tube is labeled with an animal number and date of sample collection. Blood samples are sent to Aptimmune Biologics, Inc. in a cooler without any ice packs (keep ambient out of light).

At Day 0 afternoon, per random assignment of pigs to groups A or B, pigs are treated with 1 mL of treatment intra-nasally. All animals are placed into the same pen, or if divided between 2 pens, 4 treated animals from each group are randomly allocated to one of the 2 pens. Blood samples are sent to Aptimmune Biologics, Inc. in a cooler without any ice packs (keep ambient out of light).

At Day 1 morning, targeting 12-18 hours post-treatment, the general health of study pigs is observed and any unusual observations are recorded if detected. A 10 mL blood sample is collected into Heparin tubes (green top). Each tube is labeled with an animal number and date of sample collection. Blood samples are sent to Aptimmune Biologics, Inc. in a cooler without any ice packs (keep ambient out of light).

At Day 3 morning, the general health of study pigs is observed and any unusual observations are recorded if detected. A 10 mL blood sample is collected into Heparin tubes (green top). Each tube is labeled with an animal number and date of sample collection. Blood samples are sent to Aptimmune Biologics, Inc. in a cooler without any ice packs (keep ambient out of light).

Results/Analysis

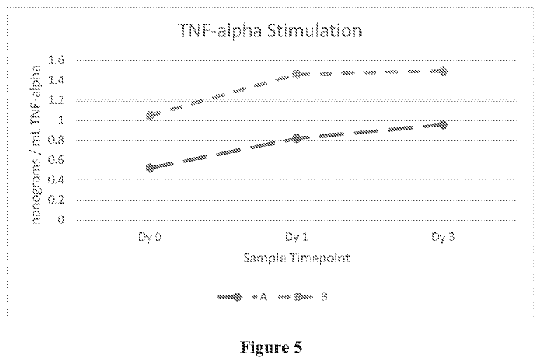

The data and results of TNF-alpha Stimulation are shown in TABLE 5 and FIG. 5. The data is shown in nanograms/mL of TNF-alpha.

TABLE 5

| | Day 0 | Day 1 | Day 3 |
|---|---|---|---|
| A | 0.521125 | 0.81675 | 0.955875 |
| B | 1.0475 | 1.4605 | 1.493 |

An increase in TNF-alpha Stimulation in group B compared to group A is observed. The results indicate that the Mycobacterium smegmatis WCL component in group B has affected the output of TNF-alpha in exposed pigs.

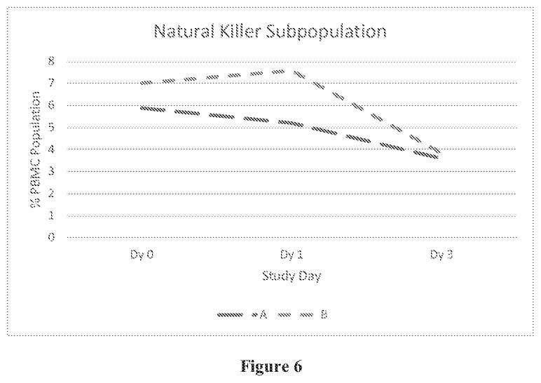

The data and results of Natural Killer Subpopulation are shown in TABLE 6 and FIG. 6. The data is shown in percent (%) of peripheral blood mononuclear cells (PBMC) Population.

TABLE 6

| | Day 0 | Day 1 | Day 3 |
|---|---|---|---|
| A | 5.87375 | 5.1925 | 3.585 |
| B | 7.00375 | 7.57 | 3.8225 |

An increase in Natural Killer Subpopulation in group B is observed at Day 1. While not wishing to be bound by any theory, it is hypothesized that a systemic immune-stimulating effect occurred in response to inoculation with the Mycobacterium smegmatis WCL component in group B.

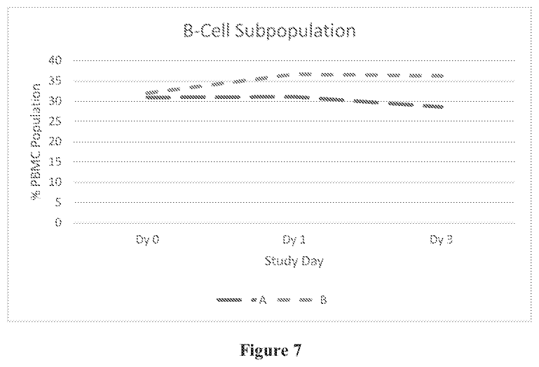

The data and results of B-Cell Subpopulation are shown in TABLE 7 and FIG. 7. The data is shown in percent (%) of PBMC Population.

TABLE 7

| | Day 0 | Day 1 | Day 3 |
|---|---|---|---|
| A | 30.9025 | 31.0375 | 28.54375 |
| B | 31.94 | 36.61125 | 36.19375 |

An increase in B-Cell Subpopulation in group B compared to group A is observed at Day 1.

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

¹ Dewey et al. 2006. Postweaning mortality in Manitoba swine. Can J Vet Res. 70(3): 161-167; Vansickle. 2006. National HogFarmer. Mortality Rates Inching Upward. http://nationalhogfarmer.com/mag/farming_mortality_rates_inching.
² Holtkamp, D., and J. Kliebenstein. 2011. PRRS Costs Industry $664 Million Annually. Pork Checkoff Study, http://www.pork.org/News/1265/PRRS CostsIndustry664Million.aspx.
³ Renukaradhya, G. J., V. Dwivedi, C. Manickama, B. Binjawadagia, and D. Benfield. 2012. Mucosal vaccines to prevent porcine reproductive and respiratory syndrome: a new perspective. Animal Health Research Review 13:1-17 doi: 10.1017/S1466252312000023.
⁴ Mateu, E., and I. Diaz. 2008. The challenge of PRRS immunology. Vet J 177:345-351, Murtaugh, M. P., Z. Xiao, and F. Zuckermann. 2002. Immunological responses of swine to porcine reproductive and respiratory syndrome virus infection. Viral Immunol 15:533-547.
⁵ Hurley, 2004, Neonatal Immune development in swine management. Proceedings of the AASV. https://www.aasv.org/library/swineinfo/Content/AASV/2004/389.pdf.
⁶ Siegrist 2007, The challenges in early life: Selected examples. J. Comp. Pathol. 137:S4-S9; Basha et al., 2015, Immune responses in neonates. Expert Rev Clin Immunol. 10(9): 1171-1184; Levy and Netea 2014, Innate immune memory: implications for development of pediatric immunomodulatory agents and adjuvanted vaccines. Pediatric Res. 75(1):184-188.
⁷ Hodgins D C, Shewen P E, 2012, Vaccination of neonates: Problem and issues. Vaccine 30:1541-1559.
⁸ Cuenca A G, Wynn J L, Moldawer L L, Levy O., 2013, Role of innate immunity in neonatal infection. Am J Perinatol. 30(2): 105-112.
⁹ Markowska-Daniel I, Pomorska-Mol M, Pejsak Z, 2011, The influence of age and maternal antibodies on the postvaccinal response against swine influenza viruses in pigs. Vet Immunol Immunopathol 142(1-2):81-6.
¹⁰ Haake M, et al. 2004. Influence of age on the effectiveness of PCV2 vaccination in piglets with high levels of maternally derived antibodies. Vet Microbiol 168:272-80.
₁₁ Che et al. 2013. Dietary plant extracts improve immune responses and growth efficiency of pigs experimentally infected with porcine reproductive and respiratory syndrome virus. J Anim Sci. 91(12):5668-79; Li et al. 2005. Effects of beta-glucan extracted from Saccharomyces cerevisiae on humoral and cellular immunity in weaned piglets. Arch Anim Nutr 59(5):303-12; Liu et al. 2013. Mannan oligosaccharide increases serum concentrations of antibodies and inflammatory mediators in weanling pigs experimentally infected with porcine reproductive and respiratory syndrome virus. J Anim Sci. 90(8):2784-93; Mao et al. 2005. Effects of beta-glucan obtained from the Chinese herb Astragalus membranaceus and lipopolysaccharide challenge on performance, immunological, adrenal, and somatotropic responses of weanling pigs. J Anim Sci. 83(12):2775-82.
¹² Geisel et al., 2005. In vivo activity of released cell wall lipids of Mycobacterium bovis Bacillus Calmette-Guerin is due principally to trehalose mycolates. J. Immunol. 174:5007; Dhiman et al., 2011. Lipoarabinomannan Localization and Abundance during Growth of Mycobacterium smegmatis. J Immunol. 193:5802.
¹³ Jollès P, Paraf A. 1973. Chemical and Biological Basis of Adjuvants. Mol. Biol. Biochem and Biophys. Vol 13. Springer-Verlag; Lederer et al. 1975. Cell walls of Mycobacteria and related organisms; chemistry and immunostimulant properties. Mol Cell Biochem 7(2): 87-104.
¹⁴ Brennan P G. 2003. Structure, function, and biogenesis of the cell wall of Mycobacterium tuberculosis. Tuberculosis 83:91-97.
¹⁵ Minnikin et al. 2015. Pathophysiological Implications of Cell Envelope Structure in Mycobacterium tuberculosis and Related Taxa, Tuberculosis—Expanding Knowledge, Dr. Wellman Ribón (Ed.), InTech, DOI: 10.5772/59585.
¹⁶ Brennan PG. 2003.
¹⁷ Bansal-Mutalik and Nikaido. 2014. Mycobacterial outer membrane is a lipid bilayer and the inner membrane us unusually rich in diacyl phosphatidylinositol diamannosides. PNAS. 111(13):4958-4963; Khoo et al. 1995. Inositol phosphate capping of the nonreducing termini of lipoarabinomannan from rapidly growing strains of Mycobacterium. J Biol Chem 270(21): 12380-9; Minnikin et al. 2015. Pathophysiological implications of cell envelope structure in Mycobacterium tuberculosis and related taxa. http://dx.doi.org/10.5772/59585)
¹⁸ Brennan and Nikaido 1995. The envelope of mycobacteria. Annu Rev Biochem. 1995; 64:29-63.
¹⁹ Ishikawa, E. et al., 2009. Direct recognition of the mycobacterial glycolipid, trehalose dimycolate, by C-type lectin. JEM 206:2879; Schoenen, H. et al., 2010. Cutting edge: Mincle is essential for recognition and adjuvanticity of the mycobacterial cord factor and its synthetic analog trehalose-dibehenate. J. Immunol. 184, 2756-2760.
²⁰ Quesniaux V J. et al., 2004. Toll-like receptor 2 (TLR2)-dependent-positive and TLR2-independent-negative regulation of proinflammatory cytokines by mycobacterial lipomannans. J Immunol. 172(7):4425-34.
²¹ Tapping R I & Tobias P S., 2003. Mycobacterial lipoarabinomannan mediates physical interactions between TLR1 and TLR2 to induce signaling. J Endotoxin Res. 9(4):264-8.
²² Lucas et al. 2015. Fractionation analysis of mycobacterial proteins. Methods Mol Biol. 1285: 47-75.
²³ Dhiman et al. 2011. Lipoarabinomannan localization and abundance during growth of Mycobacterium smegmatis. J Bacteriol. 193(20):5802-9.
²⁴ Ishikawa et al. 2009.
²⁵ Jo E K. 2008. Mycobacterial interaction with innate receptors: TLRs, C-type lectins, and NLRs. Curr Opin Infect Dis. 21(3): 279-86.
²⁶ Khoo et al., 1995. The NOD2 responds to bacterial peptidoglycan-derived muramyl dipeptide (“MDP”).
²⁷ Behr M A, Divangahi M. 2015. Freund's adjuvant, NOD2 and mycobacteria. Curr Opin Micorbiol. 23:126-32.
²⁸ Jo et al. 2007. Intracellular signalling cascades regulating innate immune responses to Mycobacteria: branching out from Toll-like receptors. Cell Microbiol. 9(5):1087-98.

We claim:

1. A method of priming a Sus' immune system, the method comprising administering an effective amount of a Mycobacterial whole cell lysate to the Sus within an effective period of time after the Sus is born.

2. The method of claim 1, wherein the Mycobacterial whole cell lysate is prepared from Mycobacterium smegmatis.

3. The method of claim 1 or claim 2, wherein the Mycobacterial whole cell lysate has not undergone purification.

4. The method of any one of claims 1 to 3, wherein the Mycobacterial whole cell lysate is unfractionated.

5. The method of any one of claims 1 to 4, wherein the Mycobacterial whole cell lysate is not delipidated.

6. The method of any one of claims 1 to 5, wherein the Mycobacterial whole cell lysate is not deproteinized.

7. The method of any one of claims 1 to 6, wherein the administration is selected from the group consisting of oral, intravenous, subcutaneous, intramuscular, intraperitoneal, intradermal, intraocular, intrapulmonary, intranasal, transdermal, subdermal, topical, mucosal, nasal, impression into skin, intravaginal, intrauterine, intracervical, and rectal.

8. The method of any one of claims 1 to 7, wherein the administration is mucosal.

9. The method of any one of claims 1 to 8, wherein the administration is intranasal.

10. The method of any one of claims 1 to 9, wherein the Mycobacterial whole cell lysate is combined with a pharmaceutically acceptable carrier.

11. The method of any one of claims 1 to 10, wherein the amount of Mycobacterial whole cell lysate administered to the Sus is from about 0.00001 to about 1000 μg of Mycobacterial whole cell lysate per kg of Sus body weight.

12. The method of any one of claims 1 to 11, wherein the amount of Mycobacterial whole cell lysate administered to the Sus is from about 1 to about 500 μg of Mycobacterial whole cell lysate per kg of Sus body weight.

13. The method of any one of claims 1 to 12, wherein the amount of Mycobacterial whole cell lysate administered to the Sus is from about 1 to about 250 μg of Mycobacterial whole cell lysate per kg of Sus body weight.

14. The method of any one of claims 1 to 13, wherein the amount of Mycobacterial whole cell lysate administered to the Sus is from about 1 to about 125 μg of Mycobacterial whole cell lysate per kg of Sus body weight.

15. The method of any one of claims 1 to 10, wherein the amount of Mycobacterial whole cell lysate administered to the Sus is from about 0.0001 to about 1000 μg of Mycobacterial whole cell lysate per mL of a pharmaceutically acceptable carrier per dose.

16. The method of any one of claims 1 to 10, wherein the amount of Mycobacterial whole cell lysate administered to the Sus is from about 1 to about 500 μg of Mycobacterial whole cell lysate per mL of a pharmaceutically acceptable carrier per dose.

17. The method of any one of claims 1 to 10, wherein the amount of Mycobacterial whole cell lysate administered to the Sus is from about 50 to about 500 μg of Mycobacterial whole cell lysate per mL of a pharmaceutically acceptable carrier per dose.

18. The method of any one of claims 1 to 10, wherein the amount of Mycobacterial whole cell lysate administered to the Sus is from about 50 to about 300 μg of Mycobacterial whole cell lysate per mL of a pharmaceutically acceptable carrier per dose.

19. The method of any one of claims 1 to 18, wherein the Mycobacterial whole cell lysate is administered as a single dose.

20. The method of any one of claims 1 to 19, wherein the Mycobacterial whole cell lysate is administered as a single unit dose.

21. The method of any one of claims 1 to 18, wherein the Mycobacterial whole cell lysate is administered as a multiple dose regimen.

22. The method of any one of claims 1 to 10, wherein the volume per dose is from about 0.001 to about 50 mL per dose.

23. The method of any one of claims 1 to 10, wherein the volume per dose is from about 0.01 to about 25 mL per dose.

24. The method of any one of claims 1 to 10, wherein the volume per dose is from about 0.1 to about 10 mL per dose.

25. The method of any one of claims 1 to 10, wherein the volume per dose is from about 1 to about 5 mL per dose.

26. The method of any one of claims 1 to 10, wherein the volume per dose is from about 1 to about 2 mL per dose.

27. The method of any one of claims 1 to 26, wherein the Mycobacterial whole cell lysate is administered to the Sus from immediately after birth to about 1 hour of age.

28. The method of any one of claims 1 to 26, wherein the Mycobacterial whole cell lysate is administered to the Sus from about 1 hour to about 24 hours of age.

29. The method of any one of claims 1 to 26, wherein the Mycobacterial whole cell lysate is administered to the Sus from about 24 hours to about 1 week of age.

30. The method of any one of claims 1 to 26, wherein the Mycobacterial whole cell lysate is administered to the Sus from about 1 week to about 1 month of age.

31. The method of any of claims 1 to 26, wherein the Mycobacterial whole cell lysate is administered to the Sus from about 1 month to about 2 months of age.

32. The method of any one of claims 1 to 26, wherein the Mycobacterial whole cell lysate is administered to the Sus from about 2 months to about 3 months of age.

33. The method of any of claims 1 to 26, wherein the Mycobacterial whole cell lysate is administered to the Sus from about 3 months to about 4 months of age.

34. The method of any one of claims 1 to 33, wherein priming a Sus' immune system comprises priming white blood cells.

35. The method of any one of claims 1 to 33, wherein priming a Sus' immune system comprises priming T cells.

36. The method of any one of claims 1 to 33, wherein priming a Sus' immune system comprises priming monocytes.

37. The method of any one of claims 1 to 33, wherein priming a Sus' immune system comprises priming macrophages.

38. The method of any one of claims 1 to 33, wherein priming a Sus' immune system comprises priming alveolar macrophages.

39. The method of any one of claims 1 to 38, wherein the primed alveolar macrophages exhibit enhanced production of TNF-alpha in response to a stimulus.

40. The method of any one of claims 1 to 39, wherein the Sus is a pig.
